(19) United States
(12) Patent Application Publication
(10) Pub. No.: US 2025/0014709 A1
(43) Pub. Date: Jan. 9, 2025

(54) INTELLIGENT DESIGN METHOD OF TYPE I DIABETES VACCINE

(71) Applicant: SHANGHAI INSTITUTE FOR ADVANCED STUDY ZHEJIANG UNIVERSITY, Shanghai (CN)

(72) Inventors: Ruhong ZHOU, Stormville, NY (US); Chun CHAN, Columbus, OH (US); Yi SONG, Shanghai (CN)

(21) Appl. No.: 18/894,542

(22) Filed: Sep. 24, 2024

Related U.S. Application Data
(63) Continuation of application No. PCT/CN2024/080942, filed on Mar. 11, 2024.

(30) Foreign Application Priority Data
Mar. 16, 2023 (CN) 202310255039.7

(51) Int. Cl. G16H 20/17; G16B 15/30; G16B 30/00

(52) U.S. Cl. CPC G16H 20/17; G16B 15/30; G16B 30/00

(57) ABSTRACT

Provided is an intelligent design method of a type I diabetes vaccine. The method according to the disclosure includes following steps: performing a computer-simulated amino acid mutation design on initial type I diabetes autoantigen sequences obtained from patients with type I diabetes, accompanied with a rational design based on a structure of an HLA-polypeptide molecule-TCR ternary complex.

### CROSS-REFERENCE TO RELATED APPLICATIONS

This disclosure is a continuation of PCT/CN2024/080942, filed Mar. 11, 2024 and claims priority of Chinese Patent Application No. 202310255039.7, filed on Mar. 16, 2023, the contents of which are hereby incorporated by reference.

### TECHNICAL FIELD

The disclosure belongs to the field of biomedicine, and relates to a scheme for obtaining a novel autoantigen by modifying and optimizing polypeptide molecules in an amino acid mutation way, and taking the novel autoantigen as a development basis of a type I diabetes vaccine, and in particular to an intelligent design method of a type I diabetes vaccine.

### BACKGROUND

Type I diabetes is an autoimmune disease caused by autonomous activation of cluster of differentiation 4+ (CD4+) and cluster of differentiation 8+ (CD8+) lymphocytes. Although related antigens that directly activate CD4+ T cell immune response have not been determined, a number of potential autogenerated antigens (autoantigens) have been widely reported, including but not limited to glutamate decarboxylase 65 (GAD65), heat shock protein (HSP), zinc transporter 8 (ZnT8), pancreatic and duodenal homeobox 1 (PDX1), insulin and some newborn antigens related to autoimmune diseases. Insulin and other related polypeptide molecules have long been used as potential autoantigens for the active and controllable activation of CD4+ T cell immune response, thus realizing the efficacy of vaccines. However, insulin and several related polypeptide molecules present complicated results in experiments of activating CD4+ T cell immune response. For example, HLA-DQ8 is an over-expressed Human Leukocyte Antigen (HLA) gene in patients with type I diabetes. Researchers find that an autoantigen polypeptide molecule has complex binding conformation and states with HLA-DQ8, making it difficult to accurately determine the related binding affinity. Since the successful formation of an HLA-polypeptide molecule-T cell receptor (TCR) ternary complex is an important prerequisite for activating the CD4+ T cell immune response, the complex binding conformation of HLA-polypeptide molecule may seriously affect the evaluation of the effect of activating the CD4+ T cell immune response by the autoantigen polypeptide molecule.

Most of the existing type I diabetes vaccines are developed on the basis of insulin or other pancreatic substances, and there is no clear activation mechanism of related immune molecules, resulting in poor efficacy. However, there are many difficulties in the design of type I diabetes vaccine based on autoantigen polypeptide molecules. One of the outstanding difficulties is that it is time-consuming and laborious to measure the binding affinity of HLA-polypeptide molecule-TCR ternary complex through experiments. Moreover, due to the lack of binding affinity data, researchers find it difficult to quantify the importance and mutable space of each site of the polypeptide molecules, resulting in difficulties in rational optimization and design of immune peptide molecules.

### SUMMARY

In view of the defects of the background technology, the disclosure provides an intelligent design method of a type I diabetes vaccine. This method may accurately describe and determine overall binding conformational differences caused by single-point or multi-point mutations in a process of optimizing a design of polypeptide molecules. The disclosure provides a computer simulated and more time-saving and labor-saving method for calculating binding affinity of an HLA-polypeptide molecule-TCR ternary complex compared to experiments. Conducting a large number of amino acid mutation simulation experiments on a basis of existing autoantigens is a new way to obtain peptide molecules for effectively inducing autoimmune reactions.

The disclosure adopts a following technical scheme.

The disclosure is based on initial type I diabetes autoantigen sequences (referred to as type I diabetes autoantigen polypeptide molecules for short) obtained from patients with type I diabetes, obtains an all-atomic three-dimensional structure by a protein structure prediction method, simulates a large number of single-point, double-point and exchangeable amino acid mutations by a computer, and rapidly and accurately measures binding affinity of the autoantigen and related immune molecules, screens autoantigen polypeptide molecules with higher HLA and TCR binding affinity than known type I diabetes autoantigen, selects several advanced autoantigen sequences to conduct an experiment of tracing T cell proliferation by analyzing carboxyfluorescein succinimidyl ester (CFSE), and selects autoantigen polypeptide molecules that may effectively induce the proliferation of type I diabetes related CD4+T lymphocytes after experimental verification as immunogenic autoantigen polypeptide molecules. The obtained autoantigen may be used as a basis for development of the type I diabetes vaccine in a form of artificial synthetic peptides molecules.

As effective components of the type I diabetes vaccine, immunogenic autoantigen polypeptide molecules are presented in a form of one or more immunogenic autoantigen polypeptide molecules, or one or more polypeptide chains of immunogenic autoantigen peptide segments, or one or more polynucleotides of amino acid sequences of immunogenic autoantigen peptide segments.

The disclosure also provides a type I diabetes vaccine, where the immunogenic autoantigen is taken as an effective component of the type I diabetes vaccine, and the vaccine is capable of being used in combination with other type I diabetes drugs.

The selection and the source of the initial type I diabetes autoantigen sequences have nothing to do with whether the patients with type I diabetes are receiving treatment related to type I diabetes. An acquisition method specifically includes following steps: firstly, sequencing at least parts of genes of patients with type I diabetes; then, obtaining initial type I diabetes autoantigen sequences by gene comparison between the patients with type I diabetes and normal people.

A specific method of the above-mentioned screening of the autoantigen polypeptide molecules with higher HLA and TCR binding affinity compared with the initial type I diabetes autoantigen sequences is as follows:
1. constructing all-atomic three-dimensional structures of an HLA-polypeptide molecule-TCR ternary complex, a polypeptide human leukocyte antigen (pHLA) binary complex and a polypeptide molecule;
2. simulating dynamic states of the HLA-polypeptide molecule-TCR ternary complex, the pHLA binary complex and the polypeptide molecule by molecular dynamics;
3. performing structural characterization of dynamic conformational changes of the HLA-polypeptide molecule-TCR ternary complex, the pHLA binary complex and the polypeptide molecule;
4. defining a “binding state” and an “unbound state” of an immune molecule complex system;
5. by a free energy perturbation method, mutating an original amino acid at a specified site on the polypeptide molecule into a target amino acid on a basis of the “binding state”, and calculating system free energy obtained or consumed in this process;
6. by the free energy perturbation method, mutating the original amino acid at the specified site on the polypeptide molecule into the target amino acid on a basis of the “unbound state”, and calculating system free energy obtained or consumed in this process;
7. subtracting a free energy difference based on the “binding state” from a free energy difference based on the “unbound state” to obtain binding affinity of the polypeptide molecule to HLA and binding affinity of the pHLA binary complex formed by the polypeptide molecule of the autoantigen sequences and an HLA molecule to TCR; and
8. screening candidate polypeptide molecules with high binding affinity to the HLA and a TCR molecule.

The beneficial effect of the disclosure are as follows.

Based on the method according to the disclosure, autoantigen polypeptide molecules with higher binding affinity for HLA and TCR molecules related to type I diabetes may be successfully screened out. CFSE-based T cell proliferation experiments may prove that the autoantigen polypeptide molecules may more effectively activate CD4+ T cells to induce immune responses, so the autoantigen polypeptide molecules are suitable for the development of the type I diabetes vaccine in the form of synthetic autoantigen polypeptide molecules.

The method according to the disclosure may accurately describe and determine the overall binding conformational differences caused by the single-point or multi-point mutations in the process of optimizing the design of the polypeptide molecules. The disclosure provides the computer simulated and more time-saving and labor-saving method for calculating the binding affinity of the HLA-polypeptide molecule-TCR ternary complex compared to the experiments. Conducting a large number of amino acid mutation simulation experiments on the basis of existing autoantigens is a new way to obtain the peptide molecules for effectively inducing the autoimmune reactions.

A free energy calculation method provided by the disclosure uses a computer to model a three-dimensional structure of a protein complex from scratch (which may include HLA protein, an antigen molecule or an autoantigen molecule and TCR protein at the same time) and carries out simulation (molecular dynamics simulation) according to a basic principle of Newtonian mechanics, and calculates and obtains free energy changes caused by specified amino acid mutations by defining the binding state and the unbound state of the complex. Since the diversity of immune-related proteins and molecules far exceeds a load range of a numerical approximation method, the generalization of binding affinity numerical prediction is well solved by modeling from scratch according to gene sequencing results of the patients with type I diabetes. This method is less dependent on the existing prior data, and may simulate interaction processes of almost all immune molecules independently. In this disclosure, the binding affinity prediction results obtained by the free energy perturbation method is usually the same order of magnitude as the experimental observation, and the error of the prediction results is usually 10-20%. Based on the method according to the disclosure, bioinformatics information may be obtained from the gene sequencing results of the patients with type I diabetes and transformed into a diversified protein complex three-dimensional structure model, so that accurate and universal binding affinity prediction may be realized, thereby guiding an intelligent optimization design of autoantigen molecules.

### BRIEF DESCRIPTION OF THE DRAWINGS

The disclosure will be further explained with attached drawings.

FIG. 1 shows an all-atomic three-dimensional structure of a pHLA binary complex of a type I diabetes autoantigen polypeptide molecule. A core antigen region of the polypeptide molecule is presented in light gray, a non-antigen region of the polypeptide molecule is presented in dark gray, and an HLA molecule is presented in transparent gray.

FIG. 2 shows variation of root mean square deviation of an overall structure of the pHLA binary complex of the type I diabetes autoantigen polypeptide molecule with simulation time in molecular dynamics simulation.

FIG. 3 shows variation of root mean square deviation of a polypeptide molecular structure with simulation time in molecular dynamics simulation of the pHLA binary complex of the type I diabetes autoantigen polypeptide molecule.

FIG. 4 illustrates an analysis of solvent-accessible surface areas of the polypeptide molecule. The higher the solvent-accessible surface area of a site, the lower the buried proportion of an amino acid, and the lower the solvent-accessible surface area of the site, the higher the buried proportion of the amino acid.

FIG. 5 is a schematic diagram of a thermodynamic cycle of a free energy perturbation method. A non-mutant region of the polypeptide molecule is presented in light gray, a mutant region of the polypeptide molecule is presented in dark gray, and the HLA molecule in a binding state is presented in transparent gray.

FIG. 6 is an all-atomic three-dimensional structure of an HLA-polypeptide molecule-TCR ternary complex of the type I diabetes autoantigen polypeptide molecule. Anchor sites 6-7 of the polypeptide molecule are presented in light gray, rest areas of the polypeptide molecule are presented in gray, the HLA molecule is presented in transparent gray, a dark part on a left side of a lower part is an a chain of a TCR molecule, and a light part on a right side of a lower part is a β chain of the TCR molecule.

FIG. 7 is a schematic diagram of a thermodynamic cycle of a free energy perturbation method. A non-mutant region of the polypeptide molecule is presented in light gray, a mutant region of the polypeptide molecule is presented in dark gray, and the HLA molecule is presented in transparent gray. A dark part on a left side of an upper part of a binding state is an a chain of the TCR molecule, and a light part on a right side of the upper part of the binding state is a β chain of the TCR molecule.

### DETAILED DESCRIPTION OF THE EMBODIMENTS

The disclosure will be further explained with embodiments.

### Embodiment 1: Structural Characterization of Type I Diabetes Autoantigen Polypeptide Molecule and HLA Molecular Complex Thereof

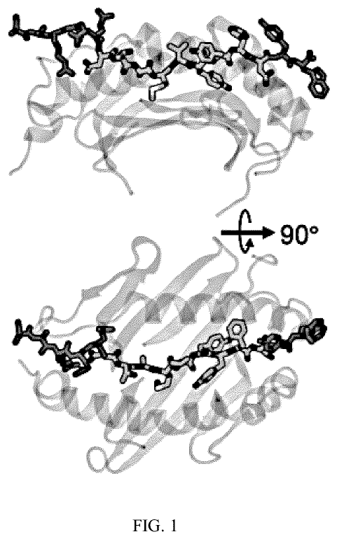

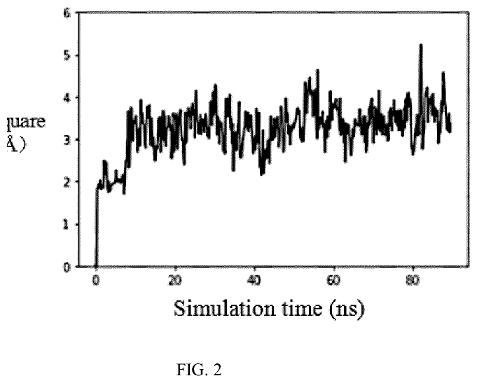

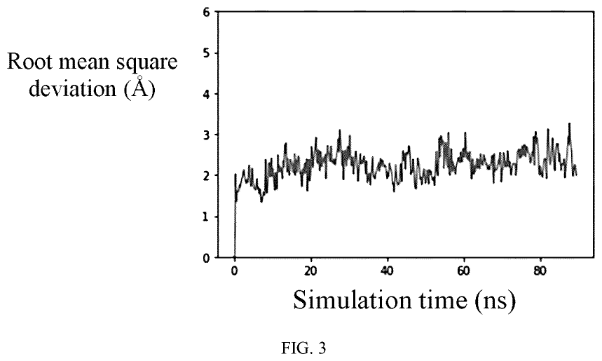

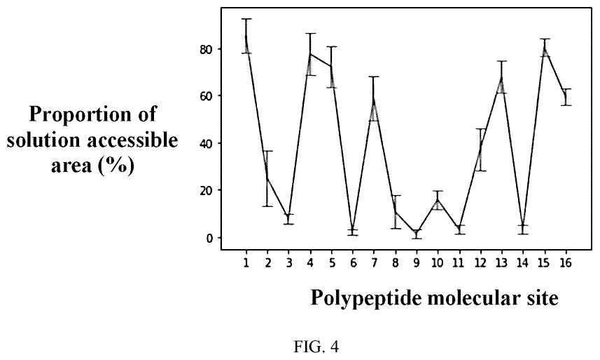

Taking an HLA-DQ8 molecule as an example, existing autoantigen polypeptide molecules that bind HLA-DQ8 are mostly 12-20 amino acids in length because of open conformation at binding sites. However, a core region that directly interacts with HLA generally contains only 9-10 amino acids, while an N-terminal and a C-terminal outside the core region of a polypeptide molecule are mostly exposed to a solution and do not directly interact with HLA. Therefore, accurately judging a solvent-accessible surface area of each site of the polypeptide molecule is helpful to preliminarily evaluate whether this site is suitable for amino acid mutation and effectively screen out sites with a high optimization success rate. A specific process is as follows:
1. determining a type I diabetes autoantigen polypeptide molecule;
2. constructing an all-atomic three-dimensional structure of a pHLA binary complex formed by the type I diabetes autoantigen polypeptide molecule and HLA by a protein structure prediction method (FIG. 1);
3. putting the type I diabetes autoantigen polypeptide molecule and the pHLA binary complex thereof into water molecules respectively, and adding ions with a same concentration as physiological ions;
4. performing molecular dynamics simulation of the system with a simulation temperature of 310 Kelvins (K), a pressure of 1 bar, a simulation step size of 2 femtosecond (fs), and a preset total number of simulation steps of 50,000,000;
5. according to root mean square deviation (RMSD) of an overall structure of the system, judging whether the structure has reached a steady state (FIG. 2);
6. according to the RMSD (especially for a binding region of the polypeptide molecule) of the system, judging whether the structure has reached a steady state (FIG. 3); and
7. analyzing a solvent-accessible surface area ratio of each site of the type I diabetes autoantigen polypeptide molecule (FIG. 4).

Combined with FIG. 1, it may be seen that a core region of the selected type I diabetes autoantigen polypeptide molecule interacts directly with HLA. By analyzing the solvent-accessible surface area, non-anchoring sites with a high proportion of buried area may be identified as potential amino acid mutation targets.

### Embodiment 2: Single Amino Acid Mutation Based on Type I Diabetes Autoantigen Polypeptide Molecule

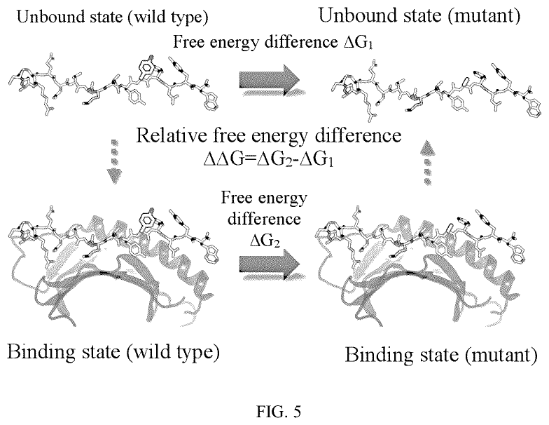

Taking the HLA-DQ8 molecule as an example, the all-atomic three-dimensional structure of the pHLA binary complex formed by the type I diabetes autoantigen polypeptide molecule and HLA is constructed (FIG. 1). By a free energy perturbation method, an original amino acid at a specified site on the type I diabetes autoantigen polypeptide molecule is mutated into a target amino acid, and system free energy differences obtained or consumed in this process are calculated, and then binding affinity of the type I diabetes autoantigen polypeptide molecule to this HLA is calculated and obtained (FIG. 5). Concrete calculation experiments include:
1. simulating a dynamic binding state of the pHLA binary complex by molecular dynamics, and defining the dynamic binding state as a “binding state”;
2. simulating a dynamic state of the type I diabetes autoantigen polypeptide molecule by molecular dynamics, and defining the dynamic state as an “unbound state”;
3. by the free energy perturbation method, mutating an original amino acid at a specified site on the type I diabetes autoantigen polypeptide molecule into a target amino acid on a basis of the “binding state”, and calculating a system free energy obtained or consumed in this process;
4. by the free energy perturbation method, mutating an original amino acid at a specified site on the type I diabetes autoantigen polypeptide molecule into a target amino acid on a basis of the “unbound state”, and calculating a system free energy obtained or consumed in this process;
5. subtracting the system free energy difference obtained based on the “binding state” from the system free energy difference obtained based on the “unbound state” to obtain a relative free energy difference; and
6. according to the relative free energy difference, calculating the binding affinity of the autoantigen polypeptide molecule obtained after mutation to HLA.

A number of single-point amino acid mutation calculation experiments are carried out for the selected type I diabetes autoantigen polypeptide molecule, so candidate autoantigen polypeptide molecules with high binding affinity for HLA-DQ8 may be screened quickly and effectively. Specific amino acid mutations include but are not limited to the following.
1. For an anchoring site of the type I diabetes autoantigen polypeptide molecule, amino acids with similar side chains are selected for single mutation.
2. For a non-anchoring site of the type I diabetes autoantigen polypeptide molecule, amino acids that are conducive to enhancing a structural freedom of the autoantigen polypeptide molecule are selected for single mutation.
3. For the anchoring site or the non-anchoring site of the type I diabetes autoantigen polypeptide molecule, with the help of prior knowledge obtained from amino acid mutation experiments of other polypeptide molecules, amino acids that may enhance the binding affinity are selected for single mutation.

A mutation strategy used by the disclosure is different from traditional bioinformatics methods, and a combination mode between the core region of the type I diabetes autoantigen polypeptide molecule and HLA is simulated through structural biology and molecular dynamics, so that amino acid mutations that may enhance HLA combination are proposed efficiently and rationally.

### Embodiment 3: Amino Acid Double Mutation or Exchange Mutation Based on Type I Diabetes Autoantigen Polypeptide Molecule

Taking the HLA-DQ8 molecule as an example, the all-atomic three-dimensional structure of the pHLA binary complex formed by the type I diabetes autoantigen polypeptide molecule and HLA is constructed (FIG. 1). By the free energy perturbation method, the original amino acid at the specified site on the type I diabetes autoantigen polypeptide molecule is mutated into the target amino acid, and the system free energy differences obtained or consumed in this process are calculated, and then the binding affinity of the autoantigen polypeptide molecule obtained after mutation to this HLA is calculated. Concrete calculation experiments include:
1. simulating a dynamic binding state of the pHLA binary complex by molecular dynamics, and defining the dynamic binding state as a “binding state”;
2. simulating a dynamic state of the type I diabetes autoantigen polypeptide molecule by molecular dynamics, and defining the dynamic state as an “unbound state”;
3. by the free energy perturbation method, mutating an original amino acid at a specified site on the type I diabetes autoantigen polypeptide molecule into a target amino acid on a basis of the “binding state”, and calculating a system free energy obtained or consumed in this process;
4. by the free energy perturbation method, mutating an original amino acid at a specified site on the type I diabetes autoantigen polypeptide molecule into a target amino acid on a basis of the “unbound state”, and calculating a system free energy obtained or consumed in this process;
5. subtracting the system free energy difference obtained based on the “binding state” from the system free energy difference obtained based on the “unbound state” to obtain a relative free energy difference; and
6. according to the relative free energy difference, calculating the binding affinity of the autoantigen polypeptide molecule obtained after mutation to HLA.

Multi-site amino acid mutation calculation experiments are carried out for the selected type I diabetes autoantigen polypeptide molecule, so candidate autoantigen polypeptide molecules with high binding affinity for HLA-DQ8 may be screened quickly and effectively. Specific calculation experiments include but are not limited to the following.
1. For the sites of the autoantigen polypeptide molecule determined in Embodiment 1, amino acid double mutation or exchange mutation is carried out.
2. A free energy decomposition analysis is carried out for results of a multi-site amino acid mutation calculation experiment.
3. Rational high-throughput amino acid mutation is carried out for an anchoring site that plays a major role in the autoantigen polypeptide molecule, so as to screen more immunogenic autoantigen polypeptide molecules.

Through the multi-site amino acid mutation calculation experiment, several groups of amino acid mutations with enhanced binding affinity are obtained. The free energy decomposition analysis determines a main contribution site in the autoantigen polypeptide molecule. Furthermore, a variety of amino acid mutations may be performed on the main contribution site in the autoantigen polypeptide molecule.

### Embodiment 4: Characterization of Influence of Amino Acid Mutation on T Cell Immune Recognition

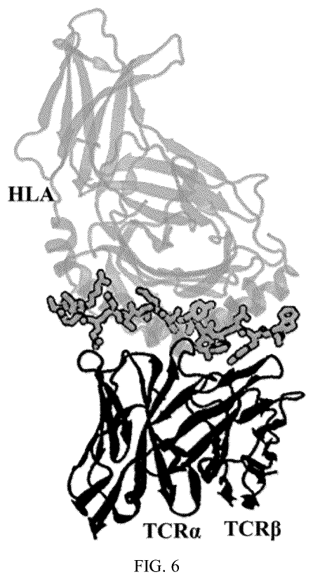

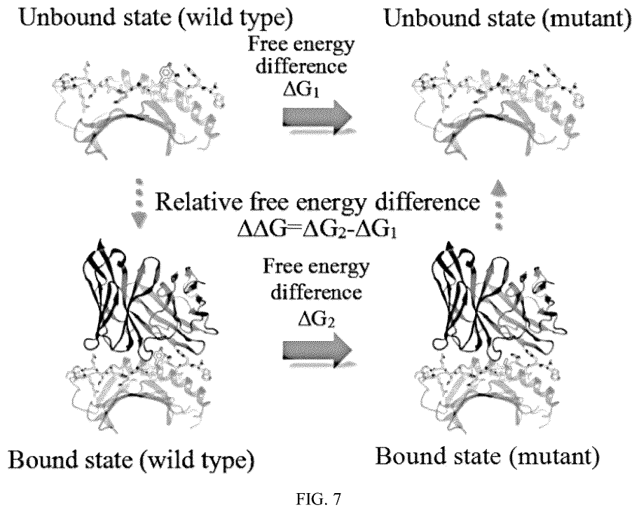

Taking the HLA-DQ8 molecule as an example, an all-atomic three-dimensional structure of an HLA-polypeptide molecule-TCR ternary complex is constructed (FIG. 6). Based on Embodiment 3, the original amino acid at the specified site on the autoantigen polypeptide molecule is mutated into the target amino acid by the free energy perturbation method, and the system free energy differences obtained or consumed in this process are calculated, and then binding affinity of TCR to the pHLA binary complex is calculated and obtained. A multi-site amino acid mutation calculation experiment is carried out for the selected type I diabetes autoantigen polypeptide molecule (as shown in FIG. 7). The concrete calculation experiment includes:
1. simulating a dynamic binding state of the HLA-polypeptide molecule-TCR ternary complex by molecular dynamics, and defining the dynamic binding state as a “binding state”;
2. simulating a dynamic state of the pHLA binary complex by molecular dynamics, and defining the dynamic state as an “unbound state”;
3. by the free energy perturbation method, mutating an original amino acid at a specified site on the type I diabetes autoantigen polypeptide molecule into a target amino acid on a basis of the “binding state”, and calculating a system free energy obtained or consumed in this process;
4. by the free energy perturbation method, mutating an original amino acid at a specified site on the type I diabetes autoantigen polypeptide molecule into a target amino acid on a basis of the “unbound state”, and calculating a system free energy obtained or consumed in this process;
5. subtracting the system free energy difference obtained based on the “binding state” from the system free energy difference obtained based on the “unbound state” to obtain a relative free energy difference; and
6. according to the relative free energy difference, calculating and obtaining the binding affinity of TCR to the pHLA binary complex.

Based on the groups of amino acid mutations with enhanced HLA binding affinity obtained in Embodiment 3, several groups are selected for multi-site amino acid mutation to calculate TCR binding affinity.

### Embodiment 5: Verification of Optimized Autoantigen Polypeptide Molecules by CFSE Tracing and Flow Cytometry Detection

By analyzing data of carboxyfluorescein succinimidyl ester (CFSE), it is verified that the optimized autoantigen polypeptide molecules may induce high lymphocyte proliferation, and with a standard gating strategy, CD4+ T cells with proliferation (low CFSE) and non-proliferation (high CFSE) may be effectively distinguished. The screened and optimized autoantigen polypeptide molecules all show that CD69 protein on surfaces of CD4+ T cells is activated, and the effect should be at least equal to that of the type I diabetes autoantigen polypeptide molecule. These optimized autoantigen polypeptide molecules may be used as effective components of the type I diabetes vaccine.

In this disclosure, the free energy perturbation method is used to calculate the binding affinity between biomolecules, and the method is essentially a free energy calculation method based on molecular dynamics simulation. Besides enthalpy calculation, the method also includes dynamic sampling and entropy calculation, and the calculation results are more accurate, as shown in Table 1. Taking an HLA-DQ8 molecule as an example, Table 1 compares the calculation results of traditional free energy calculation and the free energy perturbation method. By comparing the relative free energy differences, it may be seen that an error of the free energy perturbation method is small. A difference of the relative free energy difference of 4.1 kilocalorie (kcal)/mol is about 1000 times the difference in binding affinity, and a common experimental measurement error is about +1 kcal/mol. WT: wild type; M4I: methionine (M) at position 4 is mutated into isoleucine (I); Y6A: tyrosine (Y) at position 6 is mutated into alanine (A); Y6V_Y7V: tyrosine (Y) at position 6 is mutated into valine (V) and tyrosine (Y) at position 7 is mutated into valine (V).

TABLE 1

| | | | | | | Free energy |
|---|---|---|---|---|---|---|
| | | | | | | perturbation |
| | Traditional free energy calculation (MMPBSA) | | | | | method |
| (Unit: | | Antigen | Antigen | Free energy | Relative | Relative |
| kcal/mol) | | presenting | polypeptide | difference | free energy | free energy |
| Mutation | Compound (1) | protein (2) | molecule (3) | (1) − (2) − (3) | difference | difference |
| WT | −7217.44 ± 412.07 | −6769.99 ± 398.35 | −358.85 ± 31.48 | −88.60 ± 15.52 | | |
| M4I | −7055.21 ± 419.84 | −6616.50 ± 407.46 | −344.50 ± 28.93 | −94.22 ± 10.72 | −5.6 ± 18.9 | −0.6 ± 0.4 |
| Y6A | −7071.15 ± 400.21 | −6620.82 ± 397.11 | −352.46 ± 27.78 | −97.87 ± 13.22 | −7.5 ± 20.4 | −9.5 ± 1.3 |
| Y6V_Y7V | −7067.77 ± 418.48 | −6626.54 ± 421.06 | −349.33 ± 30.94 | −91.89 ± 13.01 | −3.3 ± 20.3 | −6.3 ± 2.8 |

What is claimed is:

1. An intelligent design method of a type I diabetes vaccine, comprising following steps:
(i) sequencing at least parts of genes of patients with type I diabetes;
(ii) carrying out gene comparison between the patients with type I diabetes and normal people to obtain initial type I diabetes autoantigen sequences;
(iii) performing a computer-simulated amino acid mutation design based on the initial type I diabetes autoantigen sequences;
(iv) analyzing and calculating HLA binding affinity of a polypeptide molecule of the autoantigen sequences obtained in step (iii), and screening autoantigen sequences with higher HLA binding affinity than the initial type I diabetes autoantigen sequences in step (ii);
(v) analyzing and calculating TCR binding affinity of a pHLA binary complex formed by a polypeptide molecule of the autoantigen sequences screened in step (iv) and an HLA molecule, and screening autoantigen sequences with higher TCR binding affinity than the initial type I diabetes autoantigen sequences in the step (ii) as candidate autoantigen sequences;
(vi) sorting immunogenicity of the candidate autoantigen sequences according to a sum of polypeptide molecule-HLA binding affinity and pHLA-TCR binding affinity, and selecting several advanced autoantigen sequences to conduct a carboxyfluorescein succinimidyl ester tracing T cell proliferation experiment, and selecting an autoantigen polypeptide molecule capable of effectively inducing proliferation of type I diabetes related CD4+T lymphocytes after experimental verification, namely an immunogenic autoantigen polypeptide molecule, and taking the immunogenic autoantigen polypeptide molecule as an effective component of the type I diabetes vaccine.

2. The intelligent design method of the type I diabetes vaccine according to claim 1, wherein an amino acid mutation in the step (iii) is to mutate one or more specific sites.

3. The intelligent design method of the type I diabetes vaccine according to claim 1, wherein in the step (iv), analyzing and calculating the HLA binding affinity of the polypeptide molecule of the autoantigen sequences obtained in the step (iii) specifically comprises: by a free energy perturbation method, mutating original amino acids at specified sites on the polypeptide molecule into target amino acids on a basis of a “binding state” and an “unbound state” respectively, calculating system free energy differences obtained or consumed in mutation processes of two states, and then calculating and obtaining the binding affinity of the polypeptide molecule to HLA, wherein the “binding state” refers to a dynamic binding state of the pHLA binary complex simulated by molecular dynamics, and the “unbound state” refers to a dynamic state of the autoantigen polypeptide molecule simulated by molecular dynamics.

4. The intelligent design method of the type I diabetes vaccine according to claim 1, wherein in step (v), analyzing and calculating the TCR binding affinity of the pHLA binary complex formed by the polypeptide molecule of the autoantigen sequences screened in the step (iv) and the HLA molecule, specifically comprises: mutating original amino acids at specified sites on the polypeptide molecule into target amino acids on a basis of a “binding state” and an “unbound state” respectively, calculating system free energy differences obtained or consumed in mutation processes of two states, and then calculating and obtaining the binding affinity of the pHLA binary complex formed by the polypeptide molecule of the autoantigen sequences and the HLA molecule to TCR, wherein the “binding state” refers to a dynamic binding state of an HLA-polypeptide molecule-TCR ternary complex simulated by molecular dynamics, and the “unbound state” refers to a dynamic state of the pHLA binary complex simulated by molecular dynamics.

5. The intelligent design method of the type I diabetes vaccine according to claim 1, wherein in step (vi), the immunogenicity of the autoantigen sequences is characterized by a CD4 response.

6. A series of immunogenic autoantigen molecules capable of being used as effective components of a type I diabetes vaccine designed based on the method according to claim 1, wherein the immunogenic autoantigen molecules are: one or more immunogenic autoantigen polypeptide molecules, or one or more polypeptide chains of immunogenic autoantigen peptide segments, or one or more polynucleotides of immunogenic autoantigen peptide segments.

7. A type I diabetes vaccine, wherein the immunogenic autoantigen according to claim 5 is used as an effective component of the type I diabetes vaccine, and the vaccine is capable of being used in combination with other type I diabetes drugs.
